(19) United States
(12) Patent Application Publication
(10) Pub. No.: US 2004/0191188 A1
(43) Pub. Date: Sep. 30, 2004

(54) Booster and activator composition for tooth-whitening agents

(76) Inventor: Brian Freedman, Weston, FL (US)

Correspondence Address: LERNER AND GREENBERG, P.A., POST OFFICE BOX 2480, HOLLYWOOD, FL, 33022-2480, US

(21) Appl. No.: 10/815,582

(22) Filed: Mar. 31, 2004

Related U.S. Application Data
(60) Provisional application No. 60/459,183, filed on Mar. 31, 2003.

(51) Int. Cl. A61K 7/20

(52) U.S. Cl. 424/53

(57) ABSTRACT

A booster and activator composition enhances tooth-whitening agents, in particular hydrogen peroxide. The composition for boosting and activating peroxides in tooth whiteners includes a heavy-metal salt solution and a chelator for preventing the heavy-metal salt solution from precipitating. The heavy-metal salt solution preferably includes manganese coordinate complexes, in particular, manganese gluconate. The chelator preferably is tetrasodudium Ethylenediamine Tetra-acetic Acid (EDTA). The composition also includes a desensitizing/adhering agent such as polyvinylpyrrolidone (PVP). The composition also includes a wetting agent such as Miranol Ultra. The composition is pH adjusted to a basic pH, preferably above 10.5.

### CROSS-REFERENCE TO RELATED APPLICATION

This application claims the benefit of U.S. Provisional Application No. 60/459,183, filed Mar. 31, 2003.

### BACKGROUND OF THE INVENTION

1. Field of the Invention

The invention relates to compositions used as boosters and activators for tooth whitening and methods of using the compositions to whiten teeth.

2. Description of the Related Art

Teeth can become discolored for many reasons. All types of tooth discoloration will combine to create a patient with a unique problem that may react very differently from person to person. Certain extrinsic food stains such as those caused by tobacco, tea, coffee, wine, and colas form by depositing chromogens directly on the tooth's surface. Different metals such as copper and nickel will cause greenish stains, while iron results in a black colored stain. Chlorhexidine and stannous fluoride can cause brown stains with a similar mechanism to that which affects apples and potatoes.

Intrinsic stains are of two types: pre- and post-eruptive. Pre-eruptive staining from fluorosis and tetracycline occurs during odontogenisis and affects the formation of the hydroxyapatite crystal. Dentinogenisis imperfecta, thalassemia, and sickle-cell anemia will also cause severe pre-eruptive staining. Teeth may be victim to post-eruptive discoloration through trauma and amalgam tattoo. Thus, our ability to effectively whiten teeth depends on a number of factors, some of which are well beyond our control.

While the base color may not be changeable, other stains have been treated by whitening agents and methods. In a 1990 study published in the Compendium of Continuing Education, Dr. David Yarborough reviewed 256 medical and dental journals to determine the safety and efficacy of the tooth whitening agents, hydrogen peroxide, and carbamide peroxide (perhydrol urea).

The evidence was conclusive that since its first use as a bleaching agent over one-hundred years ago, hydrogen peroxide, even in concentrations as high as 50%, is a safe and effective tooth whitener. At-home whiteners usually use between 2% and 10% hydrogen peroxide and tend to be quite safe, although many patients find that strengths of 8% and over, produce uncomfortable tooth sensitivity. At these strengths, some soft tissue irritation occurs, particularly when poorly fitting custom trays are used. Therefore, for at-home use, the best combination of safety and efficacy seems to be with a 7.5% gel.

In-office whiteners are available by a variety of manufacturers in strengths between 15% and 35%. Provided that adequate soft-tissue protection is employed, these are quite safe and result in no undue tooth sensitivity. In fact, S. Cohen in a 1979 Journal of Endodontics article, concluded that the majority of sensitivity during and after vital bleaching was caused by heat application. He studied patients using 35% hydrogen peroxide and 129° F. heat and concluded that 22% had no sensitivity and 78% had sensitivity lasting up to 24 hours. It is however undeniable that heat application to the whitening procedure will provide results up to 30% better and thus, in view of the minimal sensitivity side effect, the use of heat is advocated.

Most of the at-home and in-office tooth whitening available today use between 10% and 15% carbamide peroxide. These products release 3% to 5% hydrogen peroxide (the active ingredient) as well as uric, carbonic and citric acids, and ammonia. The urea is added to extend the shelf life by lowering the pH to acidic levels. However, the urea is very irritating to the teeth and, causes sensitivity. The carbopol, which is added as a thickening agent and a hindrance to hydrogen peroxide release, irritates the soft tissues.

Commercial applications of the chemistry exist. For example, Proctor and Gamble has brought the CREST WHITESTRIP® product to market. In its basic commercial form it contains 3.5% H₂O₂. At this strength, the efficacy is modest at best. The company also markets a 6.5% version that is dispensed in-office and offers a much superior whitening result.

### SUMMARY OF THE INVENTION

It is accordingly an object of the invention to provide a booster and activator composition for tooth-whitening agents that improves the heretofore-known compositions of this general type that utilize hydrogen peroxide. In addition, the composition is non-irritating, has non-acidic pH levels, is chemically simple, and has long-term safety.

With the foregoing and other objects in view, there is provided, in accordance with the invention, a composition for boosting and activating peroxides in tooth whiteners that includes a heavy-metal salt solution with a pH greater than 7, preferably between 10.3 and 10.8; and a chelator for preventing the heavy-metal salt solution from precipitating. The heavy-metal salt solution preferably includes manganese coordinate complexes, in particular, manganese gluconate. The chelator preferably is tetrasodudium Ethylenediamine Tetra-acetic Acid (EDTA). The composition also can include a wetting/adhering agent such as polyvinylpyrrolidone (PVP). Preferably, the manganese gluconate forms at least three percent of a total weight the composition.

The Chemistry of Tooth Whitening:

The whitening of teeth occurs as a result of chemical oxidation wherein organic stain molecules within the tooth structure are rendered colorless by their conversion to smaller, simpler unpigmented molecules. The organic stain molecules typically contain carbon-carbon double bonds. The oxidizing agent is H₂O₂ (hydrogen peroxide), which, under the influence of heat energy over a period of time, is itself reduced, yielding a free oxygen radical (O₂) and a stronger perhydroxyl radical (HO₂). The reducing agent is the intrinsic stain molecule in the tooth enamel that is then oxidized by these two radicals. This mechanism involves the breaking of this highly pigmented molecule at its carbon-carbon double bond. This yields two colorless hydroxyl (alcohol like) groups that reflect less light than the original larger stain molecule.

This oxidation process is further accelerated by heat.

Therefore, by the process of reduction (of the oxidizing agent—H₂O₂) and oxidation (of the reducing agent—stain molecule) in a “redox” reaction, the tooth appears whiter and whiter as more and more of the stain molecules are oxidized. This is simply because the two new smaller hydroxyl molecules reflect less light and therefore the tooth appears lighter in color.

An important factor in this process is the type of free radicals produced by the hydrogen peroxide when it is reduced. In its normal aqueous form, the peroxide chemistry produces both the weaker free oxygen radical (O₂) and the more potent perhydroxyl radical (HO₂). Evidence exists that more complete peroxide reduction and thereby the formation of more active perhydroxyl radicals can be achieved by the addition of certain “Boosters” to the tooth surface before the peroxide is applied. As a result, the number of stain molecules that are oxidized and rendered colorless is increased. This thereby achieves a greater whitening result in the same time and with the same strength of hydrogen peroxide.

Beyond the use of “Boosters” to enhance the whitening power of H₂O₂, various chemical “Activators” can be included to increase further the tooth whitening efficacy of H₂O₂ by a factor of 1.5 to 8 times.

The combination of a booster and activator in one simple product to be used in conjunction with H₂O₂ is an ideal way to achieve even whiter teeth, quicker and with no undesirable side effects.

Generally, the booster-activator should include a heavy metal salt. In particular, the heavy metal salt can be a coordinate of a manganese coordination complex compound, in particular, mangangese gluconate.

However, unless somehow chelated, manganese gluconate will precipate when in an alkaline solution; this makes it useless. To prevent this, the invention proposes to include a chelating agent such as tetrasodium EDTA.

The pH of the composition can be adjusted using most bases. NaOH is an example of a suitable base.

Polyvinylpyrrolidone (PVP) can be added to the composition. The PVP densensitizes the teeth as well as well as helps the composition to cling to the patient's teeth.

Formulations containing the composition of peroxide, booster, and activators can be applied in the following way. The product is applied to the tooth surface before the peroxide and alters the environment of the redox reaction thereby promoting the whitening reaction.

In accordance with a further object of the invention, the strongest possible hydrogen peroxide chemistry is to be used for both in the office and home protocols. Maximizing the hydrogen peroxide chemistry ensures the greatest number of free radicals for the oxidation process and thus renders a maximum number of stain molecules colorless.

Evidence indicates that when existing peroxides are used in conjunction with a power (i.e. Booster and Activator) protocol, both as a pre-whitener and as a maintenance product, the long-term results are dramatically improved. There is also ample evidence that this product could be even more effective, with no sensitivity side effects, at 7.5% and if the patients kept their mouths closed to increase the effect of body heat on the reaction. Also applying the peroxide chemistry to a clean dry tooth surface would be of clinical value and enhance results.

The addition of a “Booster-Activator” (BA) to power tooth whitening utilizing peroxides would improve results.

In accordance with a further object of the invention, applying heat to an even stronger peroxide (15%) along with BA and tissue protection would permit a patient could then wait in the waiting room while their teeth were whitened to the absolute lightest possible shade. This procedure would be welcomed by the dental community since no investment or chair time would be necessary and the revenue stream would equal the “light-activated” systems.

Yet another factor in the process, is the presence of decomposition catalysts and the enzyme salivary peroxidase, found in saliva, which will change the reduction and ionization of the H₂O₂ so that no free radicals are produced—only H₂O and O₂. These chemicals are an important part of the body's defense mechanism against oxygen toxicity. Thus, the surface of the tooth must be kept absolutely dry and free of all debris while whitening the teeth. Otherwise, the hydrogen peroxide will be rendered ineffective as an oxidizing agent.

“Anti-oxidant” vitamins (i.e. vitamins A, C, and E) act as free-radical “scavengers”. Since an excess of free oxygen and perhydroxyl radicals is one of the causes of premature skin aging as well as other medical problems, their vitamin-assisted removal can help users feel and appear younger. However, with regard to teeth, the oxidation process is a desirable and in fact necessary part of the whitening process.

Therefore, in summary, the following factors that result in much whiter teeth are sought to be optimized:

1) Using hydrogen peroxide rather than carbamide peroxide.

2) Strength of the oxidizing agent should be 35% for in-office formulations.

3) Use up to 7.5% peroxide in at-home protocols.

4) Maintaining surface cleanliness and dryness of the teeth being whitened.

5) Use of a “Booster-Activator” to increase whitening efficacy.

6) Increasing the temperature of the whitening environment as high as possible.

7) Increasing the time of tooth exposure for both the chemistry and heat.

To heat the teeth without damaging the surrounding gum tissue, the following methods are possible. After application, a possible heating step involves a minimum of three thirty-second xenon light passes per tooth. In an alternative step, a maximum of three fifteen-minute applications of 2200 mW light, which tends to produce heat, can be used. In particular, bulbs and lenses that provide to all teeth simultaneously are particularly useful, such as those sold under the trade name VIRTUOSO CRYSTAL LIGHT by Denmat.

In accordance with a further object of the invention, a home maintenance program to keep the teeth as white as possible can be offered.

By following these methods and utilizing the compositions, patients may achieve the whitest possible teeth in the least possible time with the maximum safety.

In accordance with a further object of the invention, the composition can be provided in a bottle with a sponge applicator, similar to whiteout. The applicator sponge is preferably shaped like a tooth.

Other features that are considered as characteristic for the invention are set forth in the appended claims.

Although the invention is illustrated and described herein as embodied in a booster and activator composition for tooth-whitening agents, it is, nevertheless, not intended to be limited to the details shown since various modifications and structural changes may be made therein without departing from the spirit of the invention and within the scope and range of equivalents of the claims.

The construction and method of operation of the invention, however, together with additional objects and advantages thereof, will be best understood from the following description of specific embodiments when read in connection with the accompanying examples.

### DETAILED DESCRIPTION OF THE PREFERRED EMBODIMENTS

### EXAMPLE

| REF. | | | |
|---|---|---|---|
| NO. | RAW MATERIALS | % BY WT | MASS |
| 1 | Water | 74.800 | 748.00 g |
| 2 | PVP K-30 | 1.000 | 10.00 g |
| 3 | Manganese | 3.000 | 30.00 g |
| | Gluconate | | |
| 4 | Tetrasodium EDTA, | 20.000 | 200.00 g |
| | 40% Solution | | |
| 5 | Miranol Ultra | 1.000 | 10.00 g |
| 6 | Sodium Hydroxide, | 0.200 | 2.00 g |
| | Flakes | | |
| | TOTAL | 100.000 | 1000.00 g |

Manufacturing Procedure:

Begin by weighing the water 1 and placing it into a stainless steel vessel equipped with a lightning mixer. While mixing rapidly, admix the PVP K-30 2. Mix until completely dissolved. Admix the manganese gluconate 3. Mix until completely dissolved. While mixing, slowly add the tetrasodium EDTA, 40% solution 4. Mix until the batch is clear. While mixing, slowly admix a soapless surfactant 5 such as the one sold under the tradename MIRANOL ULTRA® by the Miranol Chemical Company, Inc. of Irvington, N.J. Mix until the batch is clear. Check the pH. If the pH is below 10.3, carefully adjust to 10.5 using sodium hydroxide 6. From experience, the pH is typically 10.7, so no sodium hydroxide was required.

The resulting composition appears as a clear, non-viscous liquid. The composition is practically colorless. The composition is practically odorless. The ph ranges between 10.3 and 10.8. The viscosity is water thin. The specific gravity ranges from 1.05 to 1.10. The total microbial count is NMT 10 microorganisms per gram.

I claim:

1. A composition for boosting and activating peroxides in tooth whiteners, comprising:
a heavy-metal salt solution with a pH greater than 7; and
a chelator for preventing said heavy-metal salt solution from precipitating.

2. The composition according to claim 1, wherein said heavy-metal salt solution includes a manganese coordinate complex.

3. The composition according to claim 2, wherein said manganese coordinate complex is manganese gluconate.

4. The composition according to claim 1, wherein said chelator is tetrasodudium EDTA.

5. The composition according to claim 1, further comprising polyvinylpyrrolidone (PVP).

6. The composition according to claim 1, wherein the pH is from 10.3 to 10.8

7. The composition according to claim 3, wherein said manganese gluconate forms at least three percent of a total weight the composition.

8. The composition according to claim 1, further comprising a surfactant.

9. The composition according to claim 1, wherein said heavy-metal salt solution is aqueous.

10. A method for whitening teeth, which comprises:
applying the composition according to claim 1 to teeth;
applying a whitening agent to the teeth.

11. The method according to claim 10, which further comprises drying the teeth before the applying step.

12. The method according to claim 11, which further comprises preventing the teeth from being moistened when the composition has been applied to the teeth.

13. The method according to claim 10, which further comprises heating the composition.

14. The method according to claim 10, which further comprises heating the composition after the applying step with a light.

15. The method according to claim 14, which further comprises:
using a xenon light as the light; and
heating each tooth a maximum of three thirty-second passes with the xenon light.

16. The method according to claim 10, which further comprises limiting a weight percentage of the hydrogen peroxide to 7.5 in an at-home formula.

17. The method according to claim 10, which further comprises including at least a weight percentage of the hydrogen peroxide of 35 in an in-office formula.
